(19) United States
(12) Patent Application Publication
(10) Pub. No.: US 2020/0368521 A1
(43) Pub. Date: Nov. 26, 2020

(54) BIO-ELECTRONIC INTERFACE

(71) Applicant: Neuroloom Limited, Nottingham (GB)

(72) Inventor: Benjamin Templeton Paul, Bristol (GB)

(21) Appl. No.: 16/960,337

(22) PCT Filed: Jan. 10, 2019
(86) PCT No.: PCT/GB2019/050065

§ 371 Date: Jul. 7, 2020

(30) Foreign Application Priority Data
Jan. 10, 2018 (GB) 1800387.1

(51) Int. Cl. A61N 1/05; A61N 1/36; G06F 3/01

(52) U.S. Cl. CPC A61N 1/0529; A61N 1/0543; G06F 3/015; A61N 1/36067; A61N 1/0551

(57) ABSTRACT

The disclosure relates to a bio-electronic interface comprising: a substrate; a plurality of micro-scale tissue-engagement-structures coupled to the substrate, in which each tissue-engagement-structure has a distal end providing a base on the substrate, in which each tissue-engagement-structures comprises one or more guidance-features configured to guide the growth of a plurality of cells; and one or more interface elements associated with each of the tissue-engagement-structures, each interface element configured to couple the one or more of the plurality of cells.

The present disclosure relates to a bio-electronic interface and in particular, although not exclusively, to a neural interface including a neuronal culture.

Bio-electronic implants are used in conjunction with computer systems in the fields of research, therapeutics and human augmentation in order to provide an interface between biological host tissue and external circuitry. Such circuitry may be used in order to take measurements or readings from the host tissue, or to stimulate activity of the host tissue, such as the firing of neurons or triggering of muscle.

The implantation of conventional high resolution or high granularity bio-electronic interface implants is highly invasive in that it relates to inserting a foreign, inorganic object into the host tissue. Such an invasive procedure typically causes trauma to the host tissue which is subjected to the procedure. In addition, the formation of functional electrical connections between the inorganic implant and the host tissue may be limited, due to the disparity of material types. In some examples, the functional connection may rapidly degrade as electronic components are rejected as a foreign body, leading to their encapsulation in fibrous scar tissue by the host immune system. In such examples, interfacing with an acceptable spatial resolution may be possible only for a short time before degrading due to the nature of the encapsulation.

A further difficulty encountered in conventional bio-electronic implants relates to the desire, for certain applications, to provide a relatively high spatial resolution, highly location specific, excitation to, or signal extraction from, the host tissue. This may be the case, for example, due to the presence of inhibitory or excitatory circuits present in the host tissue with relatively small spatial separation from each other. For example, in some neuronal applications, such as in the retina of the eye or in the brain, it is desirable for an implant to be able to provide multiple parallel interfaces between the external circuitry and different locations of the target tissue which provide respective aspects of the host neuronal circuitry.

One or more embodiments herein may address the above-mentioned problems.

The listing or discussion of an apparently prior-published document in this specification should not necessarily be taken as an acknowledgement that the document is part of the state of the art or is common general knowledge.

According to a first aspect of the invention there is provided a bio-electronic interface comprising:
a substrate;
a plurality of micro-scale tissue-engagement-structures, in which each tissue-engagement-structures has a base on the substrate and comprises one or more guidance-features configured to guide the growth of a plurality of cells; and
one or more interface elements associated with each of the tissue-engagement-structures, each interface element configured to couple the one or more of the plurality of cells.

Also disclosed is a bio-electronic interface comprising:
a substrate;
a plurality of micro-scale tissue-engagement-structures coupled to the substrate, in which each tissue-engagement-structures comprises one or more guidance-features configured to guide the growth of a plurality of cells; and
one or more interface elements associated with each of the tissue-engagement-structures, each interface element configured to couple the one or more of the plurality of cells.

The plurality of micro-scale tissue-engagement-structures may comprise an array of tissue-engagement-structures.

One or more dividers may be provided on the substrate to form a plurality of cell-separation-regions associated with the plurality of tissue-engagement-structures. Each tissue-engagement-structures may have at least one cell-separation-region.

The substrate may comprise a reservoir for the plurality of cells. The substrate may comprises one or more apertures to allow cells to pass between the reservoir and a surface of the substrate on which the bases of the respective tissue-engagement-structures are provided.

The substrate may comprise a plurality of layers. A respective subset of the plurality of interface elements may be associated with each of the plurality of layers of the substrate. A respective subset of the plurality of interface elements may and opens into each of the plurality of layers of the substrate.

One or more of the tissue-engagement-structure may comprise a helical or spiral core.

The one or more guidance-features may comprise one or more cavities within one or more of the tissue-engagement-structures. The one or more cavities may each extend along the respective tissue-engagement-structures in a longitudinal direction. A divider may separate a first cavity from a second cavity within a particular, or within each tissue-engagement-structure. The divider may be hollow to allow neurites to be routed in two or three dimensions to a particular interface element. The routing may facilitate redistribution of the neurites to increase the density of the tissue engagement structures beyond that of the interface elements.

One or more of the tissue-engagement-structures may each comprise a plurality of channels, or cavities. A channel or cavity associated with a particular tissue-engagement-structure may laterally extend across the substrate by a different extent to another channel associated with the particular tissue-engagement-structure. The channels may also form the dividers of other compartments, allowing the routing of the neurites.

One or more, or each, of the tissue-engagement-structures may comprise an outer covering that surrounds a core. The outer covering may be columnar, or tubular. The outer covering may taper along the tissue-engagement-structures. The divider may be provided by a core of the particular, or each, tissue-engagement-structure. A respective interface element may be provided in, or associated with, each cavity.

Each cavity has a proximal opening and a distal opening. An opening may also be referred to as a hole or aperture. The proximal opening of the first cavity is at a different longitudinal position to the proximal opening of the second cavity. The one or more guidance-features may comprise striations or grooves within the one or more cavities. The one or more guidance-features may comprise striations or grooves on an exterior of one or more of the tissue-engagement-structures.

A first set of striations on an exterior of one of the tissue-engagement-structures may be separated from a second set of striations on that particular tissue-engagement-structure. Each tissue-engagement-structure may have a distal end providing a base on the substrate. The distal end having a width of one of 10, 25, 50, 100 microns or less. Each base may have one or more buttresses configured to support the tissue-engagement-structure that extends from the base.

The buttresses may comprise buttress-guidance-structures. The buttress-guidance-structures may be configured to guide the growth of the one or more cells towards the guidance-structures of the micro-needle. The outer surface of the tissue engagement structure may also be textured, have striations or micro-topographies which encourage the neurites to enter into the needles or dissuade the neurites from growing on the outer surface of the tissue engagement structure.

Each tissue-engagement-structure may extend transverse to the base in a longitudinal direction. Each tissue-engagement-structure may be needle-like, for example, in that it tapers and has a pointed proximal end. The tissue engagement structures may be tubular. The tissue engagement structures may be bundled together to present a surface of openings. The base of one or more of the tissue engagement structures may provide an offset in the longitudinal direction and supports a cover of the tissue engagement structures.

One or more, or each, of the tissue-engagement-structures may have a proximal end and may have a distal end. The proximal end of one or more, or each, of the tissue-engagement-structures may have a width of one of 1, 2, 5, 10, 25 microns or less. The tissue-engagement-structures may be micro-scale in that they have at least a proximal end of micron-scale dimensions. The proximal end of one or more, or each, of the tissue-engagement-structures may have an annular tip with an opening to the one or more cavities.

The bio-electronic interface may comprise a plurality of electrodes, optodes or chemical stimulators. Each electrode may be configured to be coupled to a respective cell or group of cells.

The bio-electronic interface may be a neural interface. The cells may be neurons. The cells may be neurons which are in a neuronal cell culture. The guidance-features may be configured to guide the growth of neurites of the neurons. The bio-electronic interface may comprise a cell culture. The one or more cells may extend along one or more of, or each, of the tissue-engagement-structures.

One or more of, or each of, the tissue-engagement-structures may be composed of degradable or partially degradable material. One or more of, or each of, the tissue-engagement-structures may be biologically inactive.

According to a further aspect there is provided a method of implanting a bio-electronic interface into a subject or an organ or a tissue, comprising:
receiving the bio-electronic interface;
introducing the substrate to the subject or the organ or the tissue;
allowing formation of connections between the plurality of cells of the interface and the subject, or the organ, or the tissue.

The introduction may be performed ex vivo.

According to a further aspect there is provided a method of manufacturing a bio-electronic interface, comprising:
providing a substrate;
forming a plurality of micro-scale tissue-engagement-structures, in which each tissue-engagement-structures comprises one or more guidance-features configured to guide the growth of a plurality of cells; and
associating one or more interface elements with each of the tissue-engagement-structures, each interface element configured to be coupled to one or more of the plurality of cells.

The method may further comprise applying the plurality or cells, or a cell culture comprising the plurality of cells, to the substrate. The plurality of cells, or the cell culture, may be applied during the step of forming the plurality of micro-scale tissue-engagement-structures on the substrate. In particular, when the cells are neurons, the plurality of cells, or the cell culture comprising the plurality of cells, may be applied to the substrate so that the somas of the neurons are in physical contact with the substrate. In this embodiment, the neurites of the neurons may grow from the substrate of the interface along the tissue-engagement-structures and/or guidance-features to a position(s) distal from the substrate. This configuration can be advantageous when in use as the interface may more effectively stimulate the neurons, as any stimulus may be transmitted from the substrate directly to the somas of the neurons. That is, the neuron, or cell, soma is proximal to the interface element to allow for closer coupling and effective stimulation or recording from the cell or neuron.

The cells may be neurons. The position of the neurons, or neurons within a neuronal cell culture, may be controlled using optical tweezers, soft lithography or manual micro-manipulation. The tissue-engagement-structures may be formed using two-photon lithography.

One or more embodiments will now be described by way of example only with reference to the accompanying drawings in which:

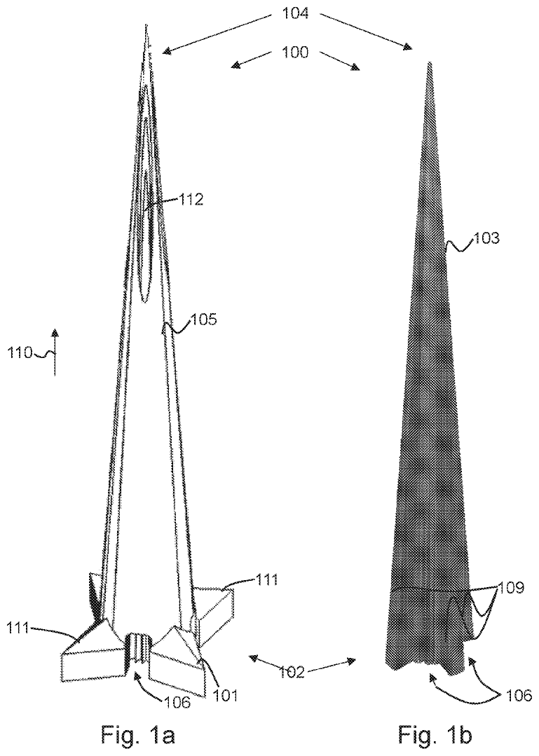

FIGS. 1a to 1c illustrate views of model tissue-engagement-structure for a bio-electronic interface;

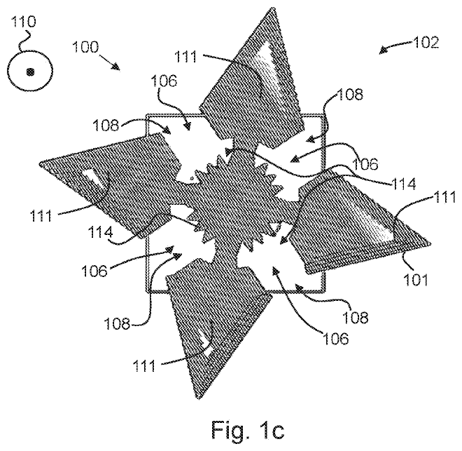

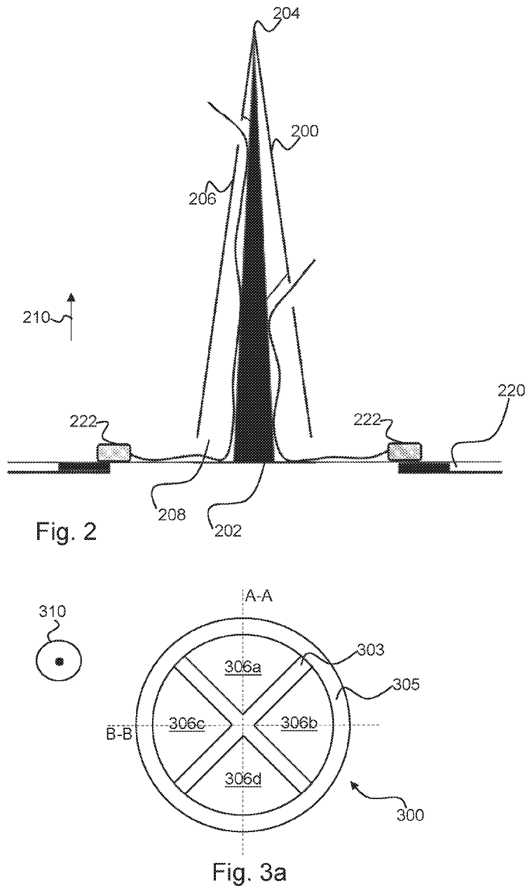

FIG. 2 illustrates a cross-section plan view of a schematic representation of a tissue-engagement-structure provided on a substrate with cells;

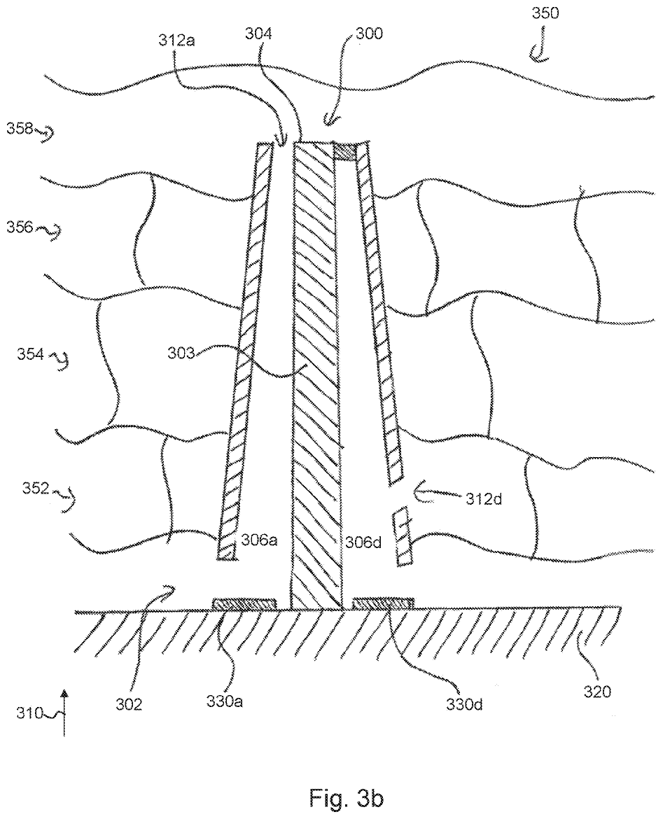

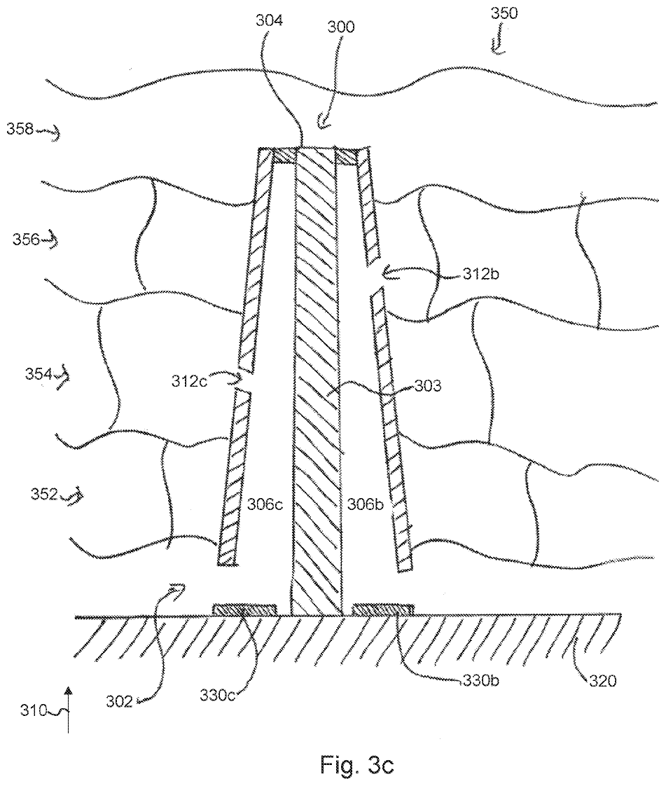

FIGS. 3a to 3c illustrate schematic representations of a tissue-engagement-structure that is engaged with tissue;

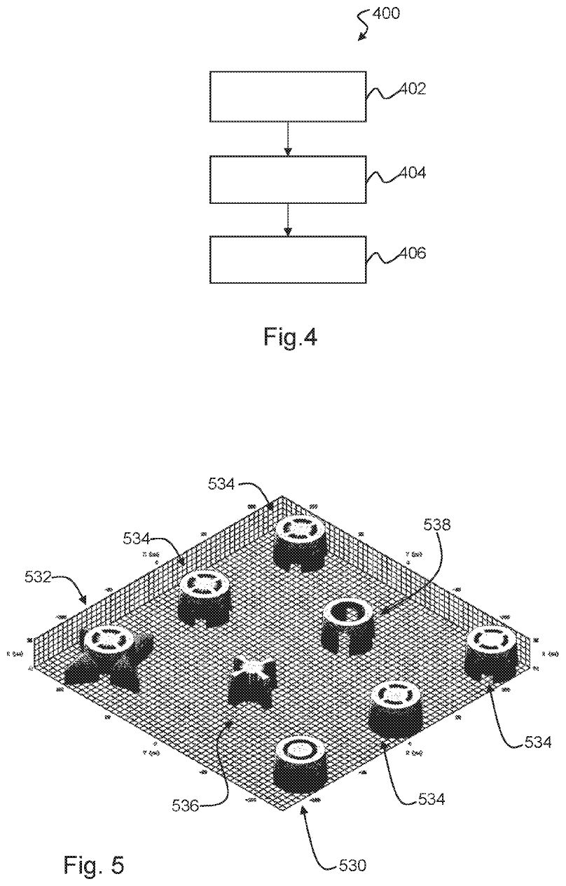

FIG. 4 illustrates a method of fabricating a bio-electronic interface;

FIG. 5 illustrates an isometric view of a cross-section of model bio-electronic interface;

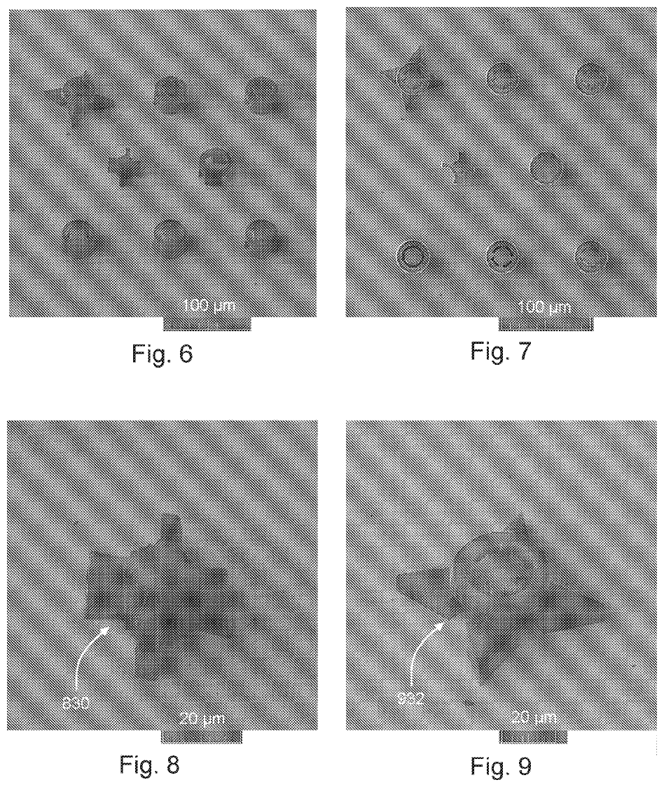

FIG. 6 illustrates, in a scanning electron micrograph (SEM), an isometric view of a fabricated structure corresponding to the design illustrated in FIG. 5;

FIG. 7 illustrates a micrograph showing a plan view of the structure illustrated in FIG. 6;

FIG. 8 illustrates a zoomed in portion of a partial tissue-engagement-structure shown in the micrograph of FIG. 6;

FIG. 9 illustrates a zoomed in portion of another partial tissue-engagement-structure shown in the micrograph of FIG. 6;

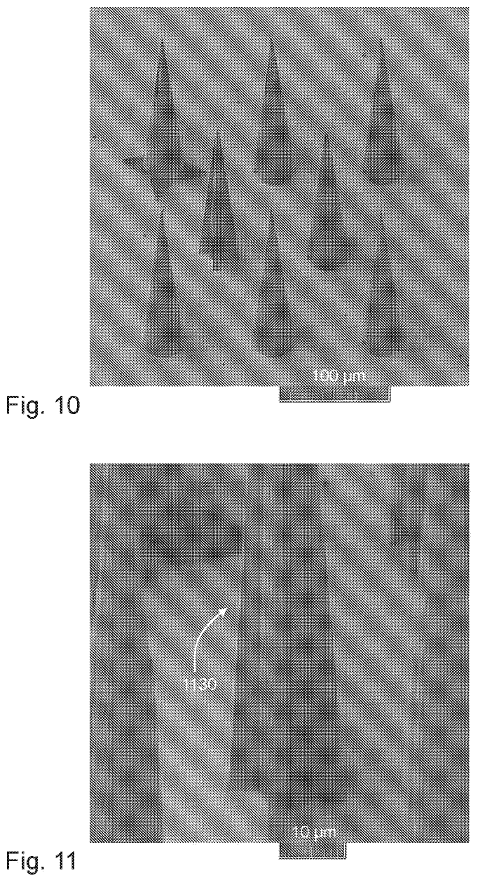

FIG. 10 illustrates a micrograph showing of an isometric view of the final structure prepared using the full design that is partially illustrated in FIG. 5;

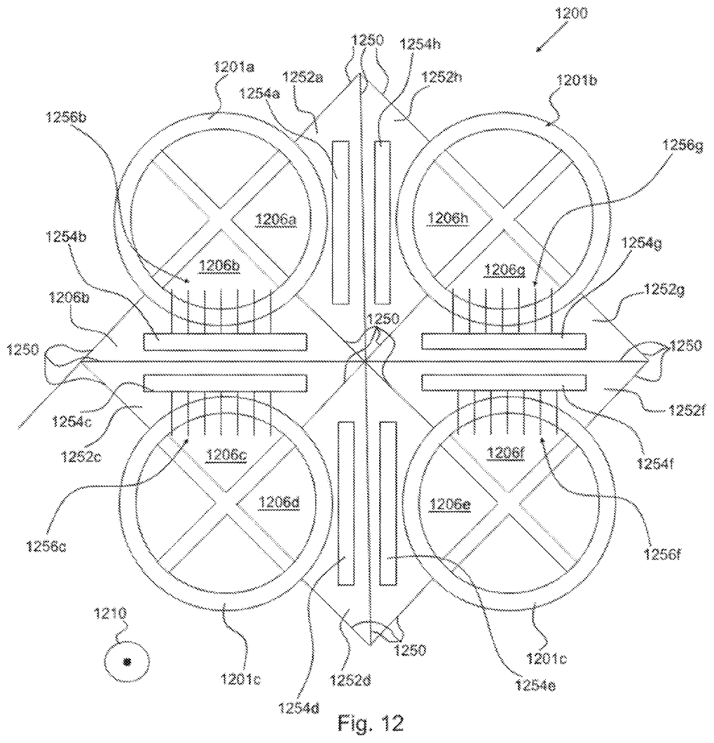

FIG. 11 illustrates a zoomed-in portion of the micrograph of FIG. 9 illustrating further detail of a tissue-engagement-structure;

FIG. 12 illustrates a cross section taken parallel to a plane of a substrate of another bio-electronic interface;

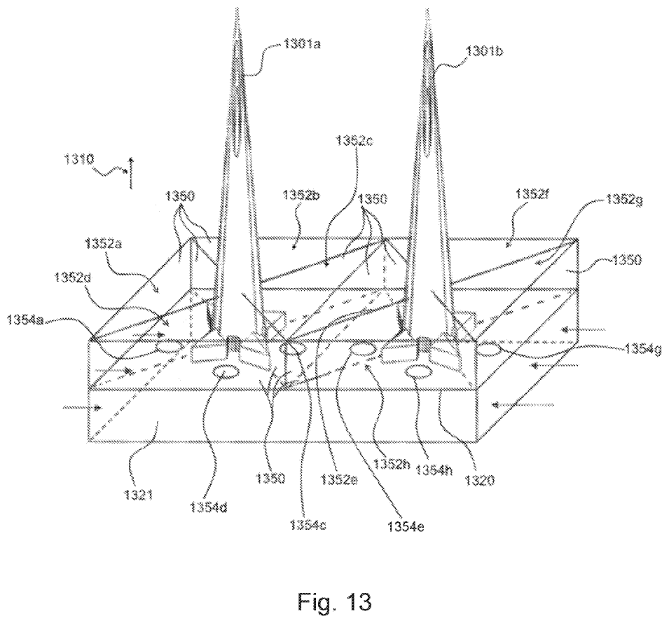

FIG. 13 illustrates an isometric perspective view of a portion of another bio-electronic interface with dividers extending in a longitudinal direction from a surface of a substrate;

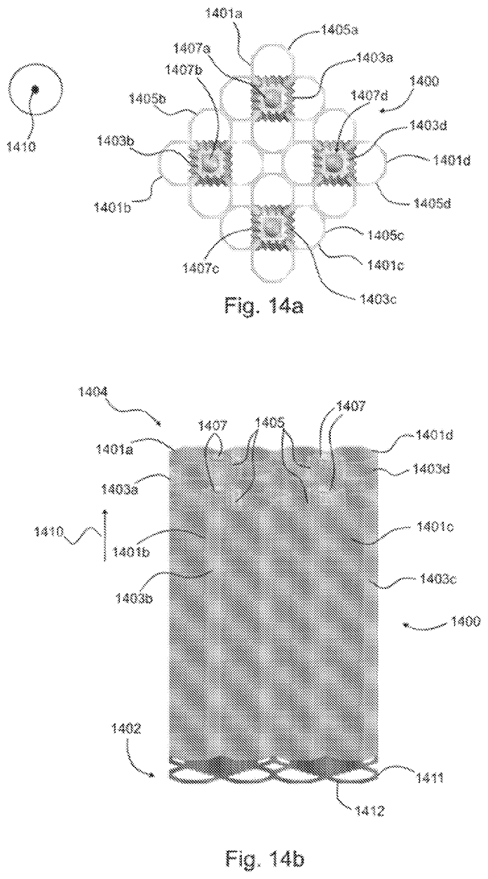

FIGS. 14a and 14b illustrate views of another plurality of tissue-engagement-structures;

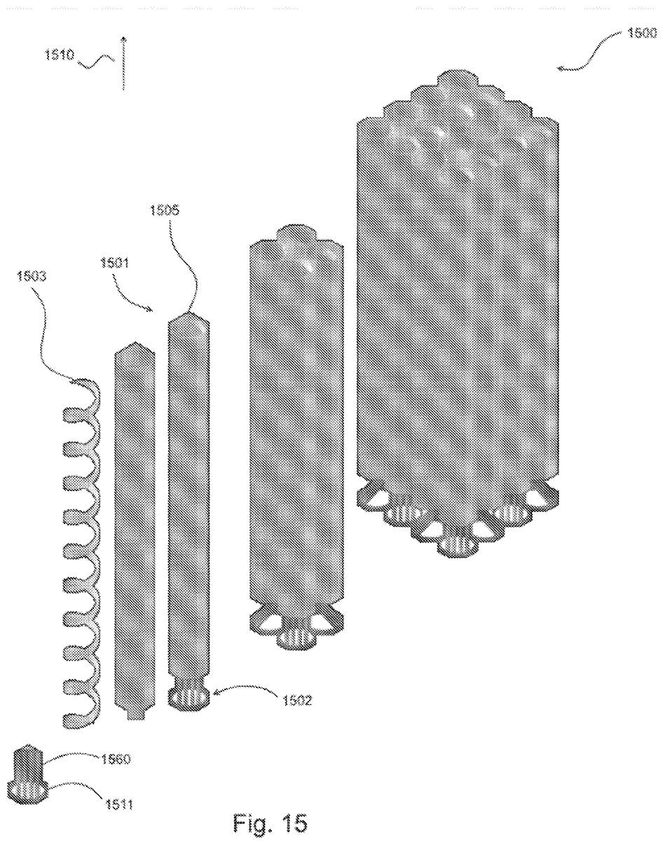

FIG. 15 illustrates a further plurality of tissue-engagement-structures;

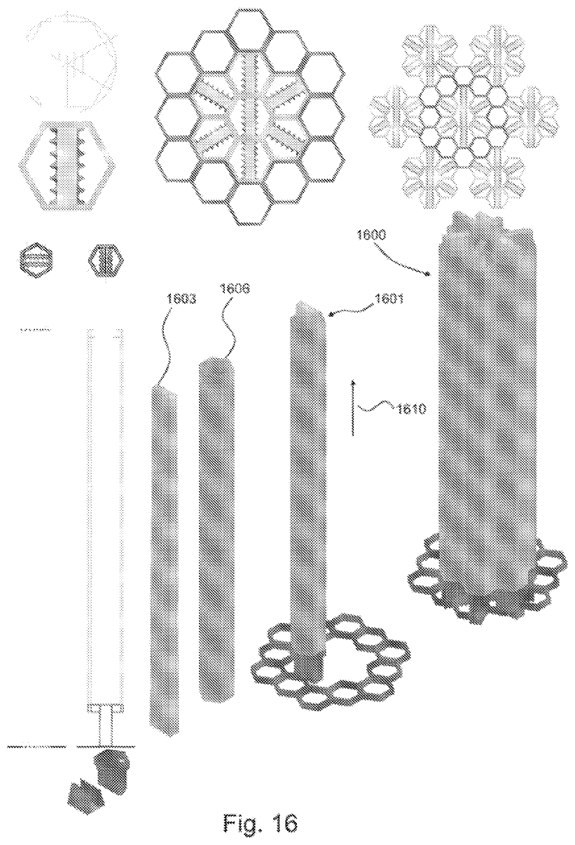

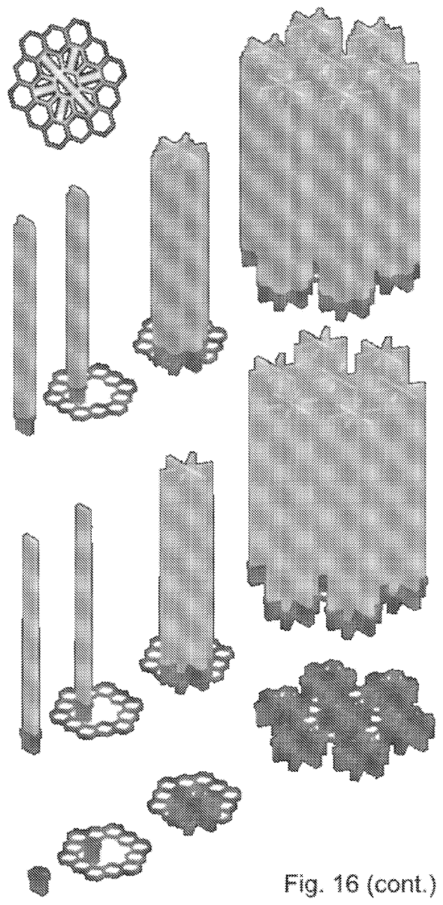

FIG. 16 illustrates various views of yet a further plurality of tissue-engagement-structures;

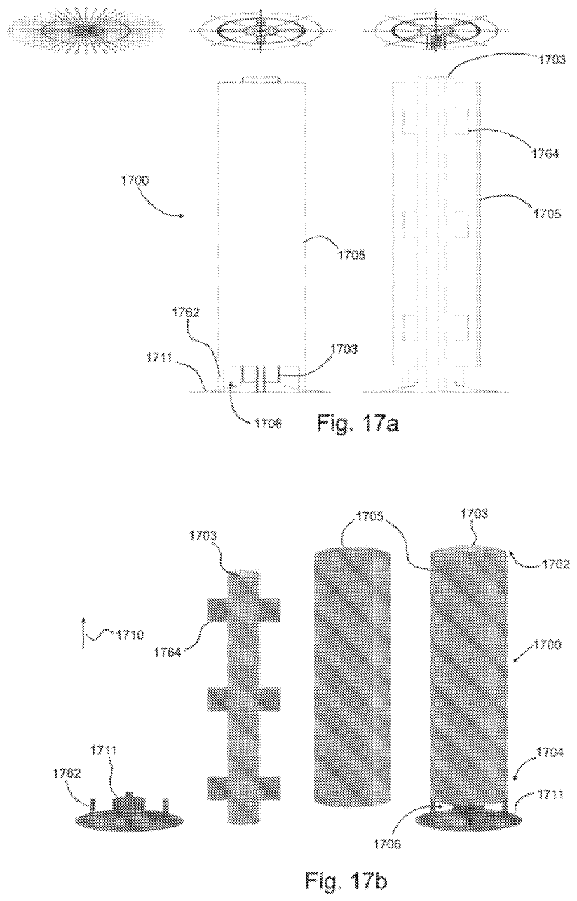

FIGS. 17a and 17b illustrate a further example of a tissue-engagement-structure in which the outer cover is offset from the substrate by a base with a plurality of openings;

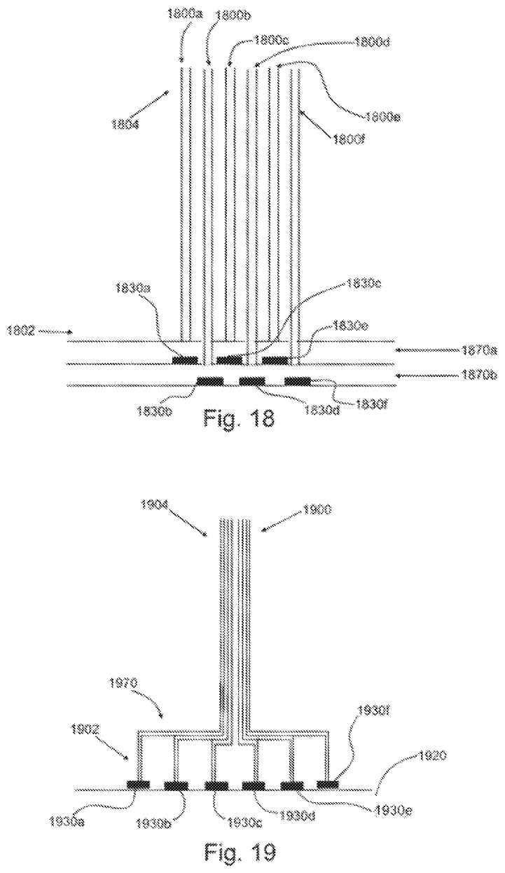

FIG. 18 illustrates an example of yet another plurality of tissue-engagement-structures; and

FIG. 19 shows another example tissue-engagement-structure of a bio-electronic interface in which an aperture density at the proximal end of the tissue-engagement-structure is greater than a number density of interface elements provided by a substrate at the distal end of the tissue-engagement-structure.

The disclosure generally relates to a bio-electronic interface comprising:
a substrate;
a plurality of micro-scale tissue-engagement-structures coupled to the substrate, in which each tissue-engagement-structures comprises one or more guidance-features configured to guide the growth of a plurality of cells; and
one or more interface elements associated with each of the tissue-engagement-structures, each interface element configured to couple the one or more of the plurality of cells. In one embodiment, the plurality of cells is in a cell culture. A cell culture may comprise the plurality of cells and a cell media, or the plurality of cells and a growth substance, or the plurality of cells and a cell media and a growth substance.

A suitable cell media for use as part of this invention, in particular when the cells are neurons, is Neurobasal (Thermo Fisher Scientific). A suitable growth substance (i.e. a supplement of growth factors) is GIBCO B27 (Thermo Fisher Scientific). A combination of Neurobasal and GIBCO B27 is particularly effective for culturing dissociated primary neurons from embryonic, or post-natal rat or mouse neurons. The growth substance or media may be adapted according to the cells cultured and may be used to influence the differentiation and maturation of the cultured cells.

Examples of interest include where the bio-electronic interface is a neural interface. In such examples, the cells are neurons or a neuronal culture. The plurality of cells or the cell culture may be considered to be part of the bio-electronic interface when the device is prepared for use as a “living implant”. Such cells or pre-formed cell cultures act as transducer between the interface elements (such as electrodes or optrodes/optode, or chemical or magnetic stimulators) and a tissue or organ of a subject that is engaged with the bio-electronic interface. In this way, the bio-electronic interface enables microelectronic systems to record or stimulate the neurons and subsequently the nervous system using a minimally invasive conduit to deliver axonal and dendritic projections from neurons to the target tissue with high spatial specificity. Such a living implant bio-electronic interface addresses the need for bioelectronic implants with higher granularity, resolution, longer term stability and cause minimal trauma or inflammatory responses, as is the case with some current solutions. These implants may be used for research, therapeutics and in human augmentation.

Another major benefit of the living implant is decoupling of electrical stimulation from the host nervous system due to the cultured neurons/cells acting as a bridge between external electronics and host tissue. Therefore, less current needs to be used due to the tight coupling of the neurons with the electrodes due to the proximity of their soma to the electrode. This is advantageous because large currents are a major cause of cell/neuron damage, which can cause scarification and loss of the input and output of an interface, as well as leading to electrolytic degradation of electrode materials in high osmolarity solutions.

In one embodiment, the neurons can be neurons of the peripheral nervous system and/or neurons of the central nervous system. In particular embodiments, the neurons are sensory neurons, and/or motor neurons, and/or interneurons, and/or neurons derived from a neuronal cell line, and/or differentiated neuron cells. Sensory neurons convert stimuli from specific receptors into action potentials, which often relate to a subject's senses (which include smell, taste, vision, hearing, and touch). The following non-limiting examples of sensory neurons are encompassed by the invention: olfactory receptor neurons (which relate to smell); glossopharyngeal nerves and chorda tympani (both of which relate to taste); photoreceptor cells (including rod cells and cone cells), bipolar cells of the retina, retinal ganglion cells, and horizontal cells (all of which relate to vision); inner hair cells and auditory nerves (both of which relate to hearing); and free nerve endings (which relate to touch; including pain). Motor neurons generally connect the central nervous system (such as the spinal cord) to other tissues or organs (such as muscles). Non-limiting examples of motor neurons are the upper motor neurons and lower motor neurons (including alpha motor neurons, beta motor neurons, and gamma motor neurons). Interneurons connect other neurons together. A non-limiting example of an interneuron is an amacrine cell, which is a type of cell found in the eye and which is involved in vision. The neuronal cell line may be an immortalised neuronal cell line. The differentiated neuronal cells may be cells that have differentiated into neurons, but which do not possess all of the characteristics of the corresponding neurons found in vivo.

It will be appreciated that one or more different cell type (such a one or more different type of neurons, e.g. sensory neurons, and/or motor neurons, and/or interneurons, and/or neurons derived from a neuronal cell line, and/or differentiated neuron cells) could be used together on the interface. As particular types of neurons will innervate different targets, an advantage of using different types of neurons is that the resulting interface will be capable of multiple functions, through innervating different targets.

In one embodiment, the cells of the bio-electronic interface are stem cells. It will be appreciated that any stem cell capable of differentiating into a neuron could be used as part of the invention, such as pluripotent stem cells (including induced pluripotent stem cells), embryonic stem cells, immortalised cell lines, adult stem cells, and/or neuronal stem cells. In some embodiments in which the cells are stem cells, in order for the stem cells to differentiate into neurons it is necessary for the bio-electronic interface to comprise one or more differentiation factors. Differentiation factors include molecules that promote the growth and/or differentiation of stem cells into neurons. Examples of differentiation factors encompassed by the invention include both soluble molecules and substrate (for example, extracellular matrix (ECM) and/or cell membrane) bound molecules, such as: Epidermal Growth Factor (EGF); Fibroblast Growth Factors (FGF) (including FGF2, FGF4, FGF8, and FGF10); Sonic Hedgehog (SHH); Bone Morphogenetic Proteins (BMP) (including BMP2 and BMP4); Platelet-Derived Growth Factor (PDGF) (including PDGF-AA; PDGF-AB; and PDGF-BB); Glycosaminoglycans (such as heparin sulphate); proteoglycans; ephrins; and Retinoic Acid. Differentiation factors can be applied to the bio-electronic interface as a coating and/or in the cell media or growth substance of the cell culture. Additionally or alternatively, the differentiation factors could be incorporated into the interface. The differentiation factors may be applied to the interface or incorporated into the interface in a spatial pattern (such as a gradient or striation). Spatial definition may be achieved using photopolymerisation or photoconjugation. In an alternative embodiment in which the cells are stem cells, it is not necessary to include one or more differentiation factors, for example because the location into which the bio-electronic interface is to be placed in the subject and/or organ and/or tissue contains the necessary environment to allow the stem cells to differentiate into neurons. By not fully differentiating the cells before implantation, the host tissue may guide the implanted neurons connections and differentiations for integration. If performed in young or embryonic animals this could allow for total integration of electronics into an existing nervous system. Further, total integration of electronics into an existing nervous system may also be achieved in adult animals. In particular, total integration may be effective in adult animals where the implanted neurons are naturally or synthetically sensitised to the host bodies molecular guidance cues, for example existing neural tracks in the host, or synthetically implanted guidance cues such as sources of diffusible chemoattractant molecules.

Due to recent advances made in high resolution 3D printing allows for the fabrication of tissue engagement structures capable of acting as conduits, and in understanding of molecular guidance cues which guide neuronal projections in vivo/during embryogenesis, it is conceivable that such a bio-electronic implant may achieve a granularity an specificity of stimulation/interfacing which could provide/facilitate an information transfer rates equal to those found in natural neural tissue and could last for the lifetime of the organism which receives the implant. The ability to incorporate new neural tissue and electrical components into the nervous system may allow for the creation of implants for human augmentation therapeutics. For example, in the case of congenital blindness, it may be possible that the neuroprosthesis is used as an augmentation rather than a therapeutic by adding an ability the patient did not previously have, rather than by restoring one. In another example, extrasensory visual input may be provided to a user (who may have full sight). Extrasensor visual input may include heat vision, augmented reality or computer output.

FIGS. 1a to 1c illustrate views of model tissue-engagement-structure for a bio-electronic interface. FIG. 1a shows an isometric perspective view of an exterior of the tissue-engagement-structure 100. FIG. 1b illustrates an isometric perspective view of a core 103 in the interior of the tissue-engagement-structure. FIG. 1c illustrates a plan view through a cross-section at a base 101 of the tissue-engagement-structure 100.

The tissue-engagement-structure 100 has a distal end 102 providing the base 101 for positioning the tissue-engagement-structure on a substrate (not shown). The base 101 has a plurality of buttresses 111 that are configured to support the tissue-engagement-structure 100 as it extends from the base 101. The distal end 102 may have a width of one of 10, 25, 50, 100 microns or less. The tissue-engagement-structure 100 also has a proximal end 104 that opposes the distal end 102. The tissue-engagement-structure 100 projects from the substrate in a longitudinal direction 110, which is transverse to a plane of the substrate, between the distal end 102 and the proximal end 104. The tissue-engagement-structure 100 may have a length in the longitudinal direction 110 of 250, 500, 1000 microns or less, for example. The tissue-engagement-structure may be provided by solid or hollow micro-needle-like structures with a pointed end of the needle-like structure provided at the proximal end 104. The proximal end 104 of the tissue-engagement-structure may have a width of one of 1, 2, 5, 10, 25 microns or less. The proximal end 104 may be configured to engage with subject neural tissue, across the entire peripheral and central nervous systems, such as brain or spinal tissue in a subject (in vivo) or biological sample (ex vivo). The geometry of the tissue-engagement-structure, or the array or grid arrangement of multiple tissue-engagement-structures, may be chosen based on the intended target tissue.

Reducing the size of the tissue-engagement-structure may reduce the immune response or scar tissue formation by increasing the long-term stability of the interface and reducing trauma.

The biocompatibility will be further increased by building the tissue-engagement-structure out of a degradable material allowing for the host tissue to heal with the ectopic neurons incorporated into the tissue. That is, long-term trauma and immunogenic response caused by the implant may be reduced by using degradable material to make up the conduit.

Term ‘substrate’ used herein generally encompasses any connection between, or support for, the tissue-engagement-structures 100. A substrate may be provided using an integrated circuit, for example, providing one or more interface elements, such as electrodes, associated with each of the tissue-engagement-structures. Each interface element is configured to couple external circuitry to one or more of the plurality of cells. Each interface element may be in, on, under, beside or adjacent to an associated tissue-engagement-structure.

The tissue-engagement-structure 100 has an outer cover 105, or shell, for shielding the cells within the tissue-engagement-structure 100 during penetration into host tissue. A core 103 is provided within the outer cover 105 which, among other things, improves structural stability of the tissue-engagement-structure 100.

In this example, a plurality of cavities 106 for receiving cells are provided within the tissue-engagement-structure 100. Each of the cavities 106 has a distal opening 108 provided at the base 101. The cavities 106 extend along the tissue-engagement-structure 100 in the longitudinal direction 110. Each of the cavities also has a proximal opening 112 (visible in FIG. 1a).

Longitudinally extending striations 114, or grooves, are provided within each of the cavities 106 on a central core 103 of the tissue-engagement-structure 100. The cavities 106 and striations 114 provide guidance features that are dimensioned such that they promote the growth along the tissue-engagement-structure 100 of cells, such as from a cell culture. Such a cell culture or cells may be provided on the substrate during use. In particular, it has been found that such guidance features are effective in promoting the growth of neurites in the longitudinal direction along the tissue-engagement-structures 100. Neurites exhibit a preference for growth along confined features. It will be understood that neurons (in particular neurites from neurons) behave in this manner due to work that has been undertaken in the field of neuron contact guidance. The needle-like structures can be optimised for minimising trauma of penetration, for example through models in FEA simulation or incorporation of elements, such as serrations, known from industry and nature to reduce trauma of penetration of incision. Guiding the growth of neurons using microstructured conduits allows for exploitation of the small size of neurites for minimising invasivity as well as guiding the projections with spatial specificity. In this way, the neurites can be targeted to microscale regions within a target tissue, allowing for higher granularity of stimulation or measurement than previously possible using some techniques, and with greater long-term stability than other high-resolution interfaces, for example, in-organic or polymer penetrating electrodes or microelectrode patch clamps.

In some examples, the striations 114 may have a groove depth or width of 500 nanometres to 1 micron for example in order to promote the growth of neurites. Sharp pointed features at the openings 108, and within the cavities 106, also provide sites for preferential neurite attraction and growth.

In this example, each of the plurality of cavities 106 is isolated from the other cavities 106 by a divider 109 provided by the core 103 of the tissue-engagement-structure 100. As such, isolated sets of one or more cells may be provided within the tissue-engagement-structure 100. Separate interface element, such as an electrode or optrode, may be associated with each of the cavities 106 in order to provide bio-electronic interfaces for the respective sets of one or more cells. The interface elements enable signals to be applied to the cells in order to activate the cells, or for signals to be received from activated cells.

A bio-electronic interface may incorporate a cell loading/delivery system which facilitates cells being delivered to the respective bases 101 of tissue-engagement-structures on the substrate. The same or a similar network (cell loading/delivery system) may be used to deliver nutrients or growth factors to influence the differentiation or growth of certain neurons on the substrate, for example.

FIG. 2 illustrates a cross-section plan view of a schematic representation of a tissue-engagement-structure 200 provided on a substrate 220 on which a number of cells 222 are provided. In this example, the tissue-engagement-structure 200 comprises a single cavity 206 extending between a distal end 202 and a proximal end 204 of the tissue-engagement-structure 200. Alternatively, the left hand side of the cavity (as shown in the figure) could be separated from the right hand side in order to provide a plurality of separately addressable electrical paths. A plurality of these cells 222 are shown as having grown into a distal opening 208 at the distal end 202 of the cavity 206 and extending in a longitudinal direction 210 from the distal end 202 towards the proximal end 204. In this example, the cavity itself provides a guidance feature for the growth of the cells 222.

In practical applications, such tissue-engagement-structures may be provided in an array or grid on a substrate in order to provide the bio-electronic interface. For some applications, it is preferable to provide a substantial number of tissue-engagement-structures, each having a plurality of separate interface elements, in order to provide a bio-electronic interface with high spatial resolution. For example, in a neural interface for retinal engaging applications, it is preferable to provide an electrode array with similar spatial characteristics to the underline fovea of the retina. That is, it is preferable for the interface to provide 13 microns squared resolution in an active area of 2.5 mm×2.5 mm. The highest density of cone receptors in the eye is 147,000 mm⁻².

In addition to the aforementioned retinal engaging application, the bio-electronic interface can also be used to engage (or innervate) the following tissues and/or organs: muscle tissue (such as cardiac muscle tissue and/or skeletal muscle tissue and/or smooth muscle tissue); nervous system tissue (such as central nervous system (CNS) tissue and/or peripheral nervous system tissue and/or enteric nervous system tissue); epithelial tissue; the lung(s); the heart; the eye(s) (including the retina, as outlined above); the ear(s); the tongue; the endocrine gland; and the nose.

Needles or design of device may be adapted to the individual target tissue. For example, a scan of a patient's retina may be used as a model to adapt, or personalise, the implant. Such a personalized device may improve surgical success or functional efficacy of the device by targeting areas of the retina which are structurally and functionally stable enough to support the device. In a further example, the density of tissue-engagement structures may be matched to a cell density of target tissue. Such methods may allow for a greater number of patients and ailments to be treated with a particular neuroprosthesis e.g. retinal prothesis where the retina is torn.

More than one cell can be formed into a cellular circuit to process a stimulus before it is received by the host tissue. For example, an inhibitory neuron may be stimulated to suppress activity of an excitatory neuron which is connected to the host. This is an example of a gated system. Such arrangements may be used to restore other attributes of neural tissue processing that are lost in disease or trauma. For example, the retina uses adjacent inhibition to enhance contrast.

FIGS. 3a to 3c illustrate schematic representations of a tissue-engagement-structure 300 engaged with a tissue of host cells 350. FIG. 3a illustrates a cross-sectional plan view of the tissue-engagement-structure 300. FIGS. 3b and 3c illustrate cross-sectional side views of the tissue-engagement-structure 300. The A-A and perpendicular B-B view are marked on FIG. 3a and presented in FIGS. 3b and 3c, respectively.

As illustrated in FIG. 3a, the tissue-engagement-structure 300 is similar to that described previously with reference to FIGS. 1a to 1c in that it has an inner core 303 that is surrounded by, and connected to, an outer covering 305. In this example, the inner core 303 has an ‘X’—shape when shown in FIG. 1, a cross section looking along an axial direction 310 of the tissue-engagement-structure 300. In this way, the core 303 defines a plurality of cavities 306a-d with the outer covering 305. Each of the cavities 306a-d provides a channel which extends along the tissue-engagement-structure 300 in the longitudinal direction 310. As shown in FIGS. 3b and 3c, the tissue-engagement-structure 300 has been inserted into the tissue of host cells 350 so that a length of the tissue-engagement-structure 300 in the longitudinal direction 310 extends through a depth of several layers, or strata 352, 354, 356, 358 of host cells.

Each of the cavities 306a-d extends in the longitudinal direction 310 between a distil end 302 and a proximal end 304 of the tissue-engagement-structure 300. Distil openings are shown at the distil end 302 of the tissue-engagement-structure 300 in this example, although there may be omitted or sealed in other examples. Each of the cavities 306a-d has a proximal opening 312a-d. A position in the longitudinal direction 310 of each of the proximal openings 312a-d differs for the respective cavities 306a-d so that the proximal openings are staggered at different points along the tissue-engagement-structure 300. Providing the proximal openings at different positions along the tissue-engagement-structure 300 enables a number of different cells to be activated using a single tissue-engagement-structure 300.

A first cavity 306 extends to a first proximal opening 312a at the tip of the proximal end 304 of the tissue-engagement-structure 300. The tip of the proximal end 304 of the other (second, third and fourth) cavities 306b-d is sealed. That is, an occlusion is provided in these cavities 306b-d at a position that is closer to the proximal end 304 than the respective positions of the proximal openings 312b-d of the second, third and fourth cavities 306b-d.

The second, third, and fourth cavities 306b-d have respective second, third and fourth proximal openings 312b-d, which are provided in the outer cover 305. The second opening 312b is closer to the distil end 302 than the first opening 312a. The third proximal opening 312c is closer to the distil end 302 than the second proximal opening 312b. The fourth proximal opening 312d is closer to the distil end 302 than the third proximal opening 312c. The proximal openings 312b-d may be provided on the same face or different faces (as shown) of the outer covering 305.

The cavities 306a-d are isolated from one another in that the core 303 provides a barrier between them within the outer cover 305. Each of the cavities 306a-d is provided with a respective interface element 330a-d. In this example, each interface element 330a-d is provided by the substrate 320 at the distil end 302. Cells (omitted from the views for clarity) may extended along and within the cavities 306a-d to provide a bio-electronic connection between the respective interface elements 330a-d and the respective distinct cells, or layers of cells 352, 354, 356, 358 of the host tissue 350. In this way, the tissue-engagement-structure 300 enables a plurality of bio-electronic interfaces to be provided by a single tissue-engagement-structure. Further, an array of such tissue-engagement-structures 300 enables a tissue to be addressable in three spatial dimensions.

In some examples, it may be desirable to prohibit or discourage the growth of cells along the outside of the outer cover 305. In examples in which openings 312b-d are provided at various different lengths along the tissue-engagement-structure 300, the presence of one or more cells along the exterior of the outer cover 305 may act to provide a short-circuit connection between different openings, thus reducing the spatial specificity of the tissue-engagement-structure 300 in the longitudinal direction 310. One or more striations may be provided on the tissue-engagement-structure 300 transverse to the longitudinal direction 310 in order to block the growth of cells along the tissue-engagement-structure 300 in the longitudinal direction 310. For example, one or more striations may extend around the tissue-engagement-structure 300. An overhang, rim or lip may also be provided on the tissue-engagement-structure 300 transverse to the longitudinal direction 310 in order to block the growth of cells along the tissue-engagement-structure 300 in the longitudinal direction 310. For example, an overhang may extend around the tissue-engagement-structure 300. The outer surface of the tissue engagement structure may also be textured, have striations or micro-topographies which encourage the neurites to enter into the needles or dissuade the neurites from growing on the outer surface of the tissue engagement structure.

Biological specificity can be exploited by getting cells which synapse with specific cellular subtypes or cells in a specific sub region of the tissue. Stimulation specificity may be improved by guiding neurons to form synapses with specific cellular subtypes by exploiting endogenous methods of axon guidance. This may be done by selecting a particular sub-type of neurons/stem cells or by using a particular combination of growth factors and/or differentiation factors. Exploiting other endogenous molecular pathways may be used to limit the number of connections/synapses the implanted neurons form with the host tissue. The implanted neurons may exploit existing topographical or molecular cues in the host tissue for navigating to a specific synaptic spot. As such, improved access to dorsal root ganglia via the dorsal root opening may be enabled.

FIG. 4 illustrates a method 400 of fabricating a bio-electronic interface. The method 400 comprises:
providing 402 a substrate;
forming 404 a plurality of micro-scale tissue-engagement-structures, in which each tissue-engagement-structures comprises one or more guidance-features configured to guide the growth of a plurality of cells; and
associating 406 one or more interface elements with each of the tissue-engagement-structures, each interface element configured to be coupled to one or more of the plurality of cells.

The method may further comprise applying a plurality of cells (such as neurons) or a cell culture comprising the plurality of cells (such as neurons) to the substrate. The neurons could come from a variety of sources, as outlined above, including stem cells, such as pluripotent stem cells (including induced pluripotent stem cells), embryonic stem cells, immortalised cell lines, adult stem cells, and/or neuronal stem cells. Different cell types, or genetically modifying/engineering neurons, may be used to vary i) the number of connections that a particular cell makes with the host, ii) the total area the cells innervate in the host or iii) the specific cell type that the innervated cells connect to in the host. Different cell types can also be used to deliver different types of signals, neurostimulation, neuroinhibition or neuromodulation, for example, to the host. Neuroinhibition, through the delivery of gamma-aminobutyric acid (GABA), for example, may reduce chronic pain or the spread of epileptic seizure activity. Neurostimulation, may be achieved using glutamate for restoring sensation, such as sight, touch or muscle control, such as in bladder incontinence or limb paralysis, for example. Neuromodulation may be used to help induce plasticity and assist children with learning difficulties or in neurodegenerative diseases which effect memory. For example, dopaminergic neurons may be used in the treatment of Parkinson's disease to replace the functionality of substanita nigra and restore motor coordination.

The donors may be of any species (preferably human, but alternatively ape, pig, mouse or rat) and may be modified genetically. The surface may be treated with an adhesion-promoting coating, or the substrate may be made of an adhesion-promoting material. Alternatively or additionally, the surface of the interface (in particular, the substrate) may be positively-charged, or may be treated so it becomes positively-charged. This might be advantageous as it could aid the attachment of cells to the interface. A variety of protocols for isolating and applying the various types of cells may be used. The cells may be applied to the interface as a cell culture or alone (i.e. not as part of a cell culture). The composition of the cell media (the solution they are cultured in) and the mechanical, chemical and biochemical composition of the substrate which they are grown on are factors in maintaining and influencing cell growth and maturation. Such a neuronal culture may be applied during the step of forming the plurality of micro-scale tissue-engagement-structures on the substrate. For example, in an additive manufacture process, the neuronal culture may be mixed in with additive manufacture feedstock. Otherwise, the neuronal culture can be added after the formation of the micro-scale tissue-engagement-structures on the substrate.

In the case that the plurality of cells (such a neurons) or a cell culture (such as a neuronal culture) is applied to the substrate before the formation of the tissue-engagement-structures, the tissue-engagement-structures may be formed on top of plurality of cells or cell culture and fastened using gelation, magnetism, chemical bonding or microstructure interactions, for example.

The needle-like structures may be dipped into or have a tip containing/made from a diffusible attractant molecule to guides the cells from the substrate into and along the needle-like structures.

A spatial pattern, for example a gradient or striation, of growth factors may be incorporated onto the interface or soluble environment of the cellular culture in order to influence cell growth, morphology, polarity or differentiation. Spatial definition may be achieved using photopolymerisation or photoconjugation. This may be achieved through a specific differentiation program or transfection of cultured neurons. Biological specificity can be exploited by patterning biomolecules into the growth substrate of the neural culture to influence differentiation or innervation specificity. The differentiation factors/growth factors/biomolecules (e.g. ephrins, semaphorins, that control the endogenous process of cell polarity, neurite outgrowth and synaptic specificity) could be added at a gradient (the highest concentration at the tip of the micro-structures) to promote neurite growth into and along the micro-structure, as well as deter the unwanted dendrites or axons, depending on the directionality of the interface, from entering the needle. This may be achieved in a gradient microfluidic chamber, through rapid exchange of resin formulation, or variation in laser intensity or dwell time, for example.

In the case that a plurality of cells (such as neurons) or a cell culture (such as a neuronal culture) is applied to the substrate after the formation of the tissue-engagement-structures, the position of the neurons or the neurons within the neuronal culture may be controlled using optical tweezers, soft lithography or manual micro-manipulation. Alternatively a resin with a high density of neurons could be selectively polymerised to fix the neurons in place. This would most likely be a gel-like material. A high molecular weight molecule (for example, alginate) would allow for low concentrations of monomer to be present in a photoresist formulation, allowing for a high density of neurons in photosensitive material (e.g. alginate, gelatine). Such manipulation may be used to promote the growth of the neurites (such as axons or dendrites) along the tissue-engagement-structures.

The method may be used to prepare a bio-electronic interface for innervating a volume of tens of microns cubed or less. However, producing micro structures of such dimensions places stringent requirements on the fabrication technique used. One fabrication technique that has been found to be suitable for producing such structures is two-photon lithography, an additive manufacture technique. In two-photon lithography, liquid material is cured in to a solid of a desired geometry under the stimulation of optical radiation. Preferably, the liquid material is a bio-compatible polymer feedstock. The polymer feedstock may comprise a polysaccharide, such as a polysaccharide that is modified with a cross-linkable group to allow for processing using two photon polymerisation (for example an acrylate or methacrylate). The polymer feedstock may also comprise a a synthetic material (for example poly-ethelyne glycol diacrylate or poly-hydroxyethylmethacrylate) or a natural material (for example gelatin, alginate or hyaluorinic acid), including a peptide that is synthetic (for example poly-tyrosine or a peptide engineered specifically for the encapsulation of neurons using two photon polymerisation) or a peptide that is natural (for example albumin or collagen). The polymer feedstock further may comprise materials derived from a natural extracellular matrix (for example matrigel). It will also be appreciated that the polymer feedstock could comprise a combination of materials, such as those listed above.

Impassivity of the bio-electronic interface may be further minimised using degradable, or partially degradable, biomaterial as a feedstock for manufacture of the tissue-engagement-structures. The liquid material may comprise a material which is chosen to have a curing energy, that is an energy required in order it is caused to crosslinking or polymerisation, that is equal to the energy provided by two discreet photons from a radiation source. Such selectivity enables the manufacturing technique to have a particularly high spatial resolution because curing only occurs when two photons are provided within a short time period and within a constrained spatial region. The dimensions of the spatial region are such that it is only provided at the very apex of a focal point of the incident radiation, as opposed to in a wider beam area of the incident radiation. In some embodiments, the material from which the interface is made could have a positively-charged surface. This may be advantageous as a positively-charged surface may aid in the attachment of the cells.

In general, the tissue-engagement-structures may be formed using one or more of the following processes: photolithography; stereolithography; electron-beam lithography; two-photon lithography; or other additive manufacturing. Alternatively, tissue-engagement-structures may be formed using moulding or casting. A mould may be formed using any of the aforementioned high resolution 3D fabrication techniques.

FIG. 5 illustrates an isometric view of a cross-section of a model for implementing using an additive manufacture technique such as that described previously with reference to FIG. 4. An array of tissue-engagement-structures 530, 532, 534, 536, 538 are provided on a substrate. The cross-section is taken towards the distal end of the array of micro scale tissue-engagement-structures (or portions of tissue-engagement-structures).

A core of a tissue-engagement-structure 530 is similar to that described previously with reference to FIG. 1b. In this example there is no additional exterior, or outer covering (such as that described previously with reference to FIG. 1a).

A second type of tissue-engagement-structure 532 is shown that is similar to that described previously with reference to FIGS. 1a and 1b in combination.

A third type of tissue-engagement-structure 534 is shown that is similar to that described previously with reference to FIGS. 1a and 1 b in combination, except that the base of the tissue-engagement-structure 536 does not have buttresses.

A fourth type of tissue-engagement-structure 536 is shown that is similar to the third type of tissue-engagement-structure 534, except that the core of the fourth type of tissue-engagement-structure 536 is without divider portions. A single cavity is thereby provided between the outer cover and core of the fourth type of tissue-engagement-structure 536.

A fifth type of tissue-engagement-structure 538 is shown that is similar to the outer cover of the third type of tissue-engagement-structure 534.

FIG. 6 illustrates an isometric perspective view of the scanning electron micrograph of a fabricated structure corresponding to the design illustrated in FIG. 5. Excellent agreement is found between the fabricated structure, prepared using two-photolithography and the intend to design.

FIG. 7 illustrates a plan view of the structure illustrated in FIG. 6.

FIG. 8 illustrates a zoomed in portion of the micrograph of FIG. 6 showing further detail of the first type of tissue-engagement-structure 830.

FIG. 9 illustrates a zoomed in portion of the micrograph of FIG. 6 showing the second type of tissue-engagement-structure 932.

FIG. 10 illustrates an isometric perspective view of the final structure based on the full design partially illustrated in FIG. 5. The image is formed using scanning electron microscopy.

FIG. 11 illustrates a zoomed in portion of the micrograph of FIG. 10 illustrating further details of the first type of tissue-engagement-structure 1130.

FIG. 12 illustrates a cross section taken parallel to a plane of a substrate of another bio-electronic interface 1200, the cross section including a cross section of a plurality of micro-scale tissue-engagement-structures 1201a-d. Each tissue-engagement-structure 1201a-d in the illustrated example is similar to that described previously with reference to FIG. 3a although in other examples a different tissue-engagement-structure may be provided.

A portion of the bio-electronic interface 1200 that is provided between the plurality of tissue-engagement-structures is also illustrated in FIG. 12. Each tissue-engagement-structure 1201a-d defines a plurality of internal cavities within its outer cover. Each of the cavities provide a channel which extends along the tissue-engagement-structure in a longitudinal direction 1210.

Each of the cavities may be associated with one or more interface elements (not shown). The one or more interface elements may be provided by one or more electrodes or optrodes, for example, as described previously. The one or more interface elements may be provided within the outer cover of the tissue-engagement-structure or elsewhere on the substrate outside of an envelope defined by the outer cover of the tissue-engagement-structure.

The bio-electronic interface 1200 further comprises a plurality of dividers 1250. The dividers 1250 also extend from the substrate in the longitudinal direction 1210. The dividers 1250 cooperate with the tissue-engagement-structures 1200a-d to form a plurality of cell separation regions 1252a-h on the substrate adjacent to the tissue-engagement-structures 1201a-d. Each cell separation region 1252a-h is associated with a respective cavity 1206a-h of the tissue-engagement-structures 1201a-d.

Cells may be introduced to the tissue-engagement-structure from above. In such case, the dividers 1250 forming the cell separation regions 1252a-h act as pockets for collecting cells. Alternatively, in examples in which the substrate comprises apertures 1254a-h, the cells may be introduced to the cell separation regions from below. That is, the cells may be introduced to the cell separation regions 1252a-h through the apertures 1254a-h in the substrate.

One or more cell-guidance-features 1256b, c, f, g may be provided on the substrate to guide the growth of cells from the cell separation region 1252a-h into an associated cavity 1206a-h of a particular tissue-engagement-structure 1201a-h. In the example shown, cell-guidance features 1256a-d are provided to guide the growth of cells from the apertures 1254 b, c, f, g in the substrate to cavities of the respective tissue-engagement-structures 1201a-d. The cell-guidance-features may be provided by striations or tracks of a chemical growth promoter, for example.

FIG. 13 illustrates an isometric perspective view of a portion of another bio-electronic interface with dividers 1350 extending in a longitudinal direction 1310 from a surface of a substrate 1320.

In this example, a plurality of tissue-engagement-structures 1301a-b that are similar to those described previously with reference to FIGS. 1a-c are provided on the substrate 1320. It will be appreciated that different tissue-engagement-structures may be provided in other examples.

The dividers 1350 define a plurality of cell separation regions 1352a-h on the substrate 1320. One or more apertures 1354a-h are provided in the surface of the substrate 1320 to allow the passage of one or more cells from a reservoir 1321 defined within the substrate through the surface 1320 of the substrate into the cell separation regions 1352a-h.

In this example, one or more cells may be introduced from outside of the bio-electronic interface and pass laterally through the internal cavity, or reservoir 1321, of the substrate before being introduced to the cell separation regions and subsequently into the tissue-engagement-structures 1301 a, b.

Alternatively, apertures in the surface of the substrate may be aligned with respective cavities in the tissue-engagement-structures so that cells may be introduced directly into the tissue-engagement-structures from a cavity within the substrate.

As a further alternative, the substrate need not itself contain a cavity, or reservoir 1321, but may instead be placed on top of a reservoir of cells in order to allow passage of the cells through the apertures 1354a-h in the substrate on to the surface 1320 of the substrate or into the tissue-engagement-structures 1301a-b.

In examples in which the cells are fed directly through the substrate into the tissue-engagement-structures, the dividers may not be necessary. In general, the dividers provided on the substrate outside of the tissue-engagement-structures act to prevent contact between cells associated with different interface elements on the substrate, and thus prevents short circuiting or amalgamation of signals.

FIGS. 14a and 14b illustrate views of another plurality of tissue-engagement-structures 1400. FIG. 14a illustrates a cross-sectional profile along an axial direction 1410 of the plurality of tissue-engagement-structures 1400. FIG. 14b illustrates a perspective side view of the plurality of tissue-engagement-structures 1400.

The plurality of tissue-engagement-structures 1400 comprises an interconnected set of individual tissue-engagement-structures 1401a-d. Each tissue-engagement-structure 1401a-d comprises an inner core 1403a-d and an outer cover 1405a-d. Each inner core 1403a-d has striations running along it in the axial direction 410. Further, each inner core 1403a-d comprises a chemical cell-attractant 1407a-d, which may be a chemical compound suitable for attracting the cells within the structure, at its proximal end 1404. In this example, the tissue-engagement-structures 1401a-d are connected along their length in the axial direction 1410, as opposed to previous examples in which the tissue-engagement-structures have been provided as separate structures on the substrate. The interconnection between the various tissue-engagement-structures 1401a-d improves the strength of the overall plurality of tissue-engagement-structures 1400.

The outer cover 1405a-d of the respective tissue-engagement-structures 1401a-d is provided by four semi-octagonal sections in this example. That is, each section would be octagonal if it were not for the core 1403a-d of the structure which replaces portions of the outer cover. Other shaped sections may be provided. Each section of the cover provides an enclosed channel about the core. The channel extends along the length of the core in the axial direction 1410. In this example, the channel is defined by the section extending from a first portion of the core to a second portion of the core. In this way, the outer cover 1405a-d provides a plurality of separate channels.

The outer cover 1405a-d of the respective tissue engagement structures 1401a-d is offset from the distal end 1402 of the plurality of tissue-engagement-structures 1400 such that, in the side view of FIG. 14b, the base 1411 is visible as a support for the cores 1403a-d and covers 1405a-d. The outer covers 1405a-d in this way provides an overhanging structure due to the offset. The overhang of the outer covers assists in preventing the growth of cells along the exterior of the outer cover which may otherwise degrade performance of the plurality of tissue-engagement-structures 1400 by providing a short circuit path between respective host cells. The base 1411 in this example comprises striations that are aligned with those of the respective cores 1405a-d. The base 1411 of each of the tissue-engagement-structures 1401a-d further comprises a stabilising portion 1412 which extends from the cross section of the core and in this example has the same arrangement, or cross-section, as the outer cover 1405a-d.

FIG. 15 illustrates a further plurality of tissue-engagement-structures 1500. The tissue-engagement-structures in this example have interconnected outer covers 1505 in the same way as those described previously with reference to FIG. 14. Each outer cover 1505 is provided by an octagonal cylinder. Each outer cover comprises a helical core 1503. The helical core 503 extends along the outer cover 1505 in the axial direction 1510. The helical core 1503 provides a guide to promote the growth of cells in the axial direction 1510. Each tissue-engagement-structure 1501 comprises a base portion 1511 that is similar to that described previously with respect of FIG. 14 except that, in this example, the base portion 1511 further comprises a plurality of guidance structures 1560. The plurality of guidance structures 1560 extend from an exterior of the tissue-engagement-structure 1501 at the distal end 1502 of the tissue-engagement-structure 1501 to the core 1503 of the tissue-engagement-structure 1501 in order to guide neurons from the substrate to the core 1503.

FIG. 16 illustrates various views of a further plurality of tissue-engagement-structures 1600. Each tissue-engagement-structure 1601 comprises a hexagonal-based cylindrical outer cover 1606 and a rectangular-based cylindrical inner core 1603 which abuts opposing inner faces of the hexagonal outer cover 1606. The inner core 1603 and hexagonal outer cover 1606 cooperate to define a plurality of separate channels within the tissue-engagement-structure 1601. Each channel extends along the length of the tissue-engagement-structure 1601 in an axial direction 1610. The inner core 1603 comprises a plurality of striations which act as cell guidance structures to promote the growth of cells along the tissue-engagement-structure 1601 in the axial direction 1610. The plurality of tissue-engagement-structures 1600 are interconnected in a similar manner to that previously described with reference to FIGS. 14 and 15. A number of separate pluralities of interconnected tissue-engagement-structures may be provided separately from one another on the substrate. The base of the tissue-engagement-structure 1601 provides an offset between the outer cover 1606 and substrate as previously described with reference to FIGS. 14 and 15.

FIGS. 17a and 17b illustrate a further example of a tissue-engagement-structure 1700 in which the outer cover 1705 is offset from the substrate, in the axial direction 1710, by a base 1711 with a plurality of openings 1706. The openings 1706 are defined by pillars 1762 of the base, which provide support to the outer cover 1705. The tissue-engagement-structure 1700 further comprises an inner core 1703 within the outer cover 1705. The outer cover 1705 provides a cylinder around the inner core 1703. The cylinder extends in the axial direction 1710. A number of struts 1764 are provided at a plurality of separate points along the axial direction to couple the outer cover 1705 to the inner core 1703. In this way, the inner core 1703 provides structural support to the outer cover 1705. In this example, the struts 1764 are discontinuous in the axial direction 1710 and do not form separate channels within the outer cover. The tissue-engagement-structure 1700 in this example therefore provides a single channel from its distal end 1704 to its proximal end 1702. Alternatively, the separate struts 1764 may be replaced by a plurality of dividers in order to segregate the tissue-engagement-structure into a plurality of separate channels.

In some implementations, size of the minimum interface element, for example the footprint or spacing of the electrodes, that can be achieved using a particular fabrication technique may place a limiting factor on the spatial resolution that can be achieved by the bio-electronic interface. That is, the diameter of an interface element that is required in order to make a connection with a cell may be substantially larger than the cell itself. Such limitations may be overcome or addressed by varying the arrangement of the interface elements on the substrate or by providing feeder portions to the tissue-engagement-structures, as discussed below with reference to FIGS. 18 and 19.

FIG. 18 illustrates an example in which a plurality of tissue-engagement-structures 1800a-f. The number density of openings in the tissue-engagement-structures 1800a-f for innervating cells within the tissue-engagement-structures 1800a-f with host cells, which in this example are all at the proximal end 1804 of the tissue-engagement-structures 1800a-f, is greater than the number density of interface elements 1830a-f that can be achieved on a single two-dimensional surface of the substrate due to the minimum size and spacing requirements of the individual interface elements. In order to increase the effective interface element density, the substrate comprises a plurality of layers 1870a-b in which each layer provides a subset 1830a,c,e; 1830b,d,f of interface elements. The plurality of layers 1870a-b forms a plurality of surfaces that are offset from one another in the longitudinal direction 1810. In this way, a subset of interface elements provided by each of the respective layers 1870a,b is associated with a corresponding subset of the tissue-engagement-structures 1800a-f.

A subset of tissue-engagement-structures 1800b,d,f may pass through a layer 1870a and maintain an isolation between the interior of the tissue-engagement-structures 1800b,d,f and the interface elements 1830a,c,e in that layer. Said subset of tissue-engagement-structures 1800b,d,f may open, at their distal end 1802, into another layer 1870b of the substrate containing interface elements 1830b,d,f that correspond to the sub-set of interface elements.

FIG. 19 shows another example tissue-engagement-structure 1900 of a bio-electronic interface in which the aperture density at the proximal end 1904 of the tissue-engagement-structure 1900 is greater than the number density of interface elements 1830a-f provided by a substrate 1920 at the distal end 1902 of the tissue-engagement-structure. In this example, the tissue-engagement-structure 1900 comprises a reconfiguration layer 1970 in which one or more of the channels defined within the tissue-engagement-structure extends laterally across the substrate 1920 in order to channel cells from an interface element that is outside of the footprint of the tissue-engagement-structure 1900 into the tissue-engagement-structure 1900 whilst maintaining separation from cells in other channels of the tissue-engagement-structure 1900.

The bio-electronic interface can be introduced into a subject and/or organ and/or tissue in vivo or ex vivo, such as by implantation The bio-electronic interfaces disclosed herein may be implanted in the subject and/or organ and/or tissue using a method comprising: receiving the bio-electronic interface; introducing the substrate to the subject and/or organ and/or tissue; and allowing formation of connections between the plurality of cells of the interface and the subject and/or organ and/or tissue. In a preferred embodiment, the plurality of cells of the interface are of a type which is endogenous to the tissue and/or organ, such as the plurality of cells being rod cells and/or cone cells and the organ being the eye.

Introducing the bio-electronic interface in vivo could be undertaken as part of a surgical procedure, such as wherein an incision is made in the subject to expose the organ or tissue, and the interface is implanted into said organ or tissue. Alternatively, a part of the interface (such as the guidance features) could be used to traverse the epidermis (such as the skin) or the sclera of the subject. This could lead to the interface being partially outside of the subject, such as the substrate being external to the epidermis or sclera. This may allow the interface to be introduced into the subject in a minimally invasive manner, such as without the need to make an incision. Introducing the bio-electronic interface ex vivo could be undertaken as part of an organ or tissue transplant or the provision of a prosthetic organ, prosthetic tissue or prosthetic limb, wherein the bio-electronic interface is implanted into the organ, tissue or limb prior to transplantation or the prosthesis being fitted.

As discussed herein, the bio-electronic interface can be used for medical purposes. Accordingly, in another aspect of the invention the bio-electronic interface described herein is for use in medicine. As will be appreciated, the bio-electronic interface can be used in medicine and to treat conditions which are associated with one or more neuronal deficiency.

Accordingly, in another aspect of the invention the bio-electronic interface is for use in preventing and/or treating a condition associated with one or more neuronal deficiency. To describe that aspect of the invention in a different way, the invention includes a plurality of cells for use in preventing and/or treating a condition associated with one or more neuronal deficiency,
wherein the plurality of cells is formulated as part of a bio-electronic interface comprising:
a substrate;
a plurality of micro-scale tissue-engagement-structures coupled to the substrate, in which each tissue-engagement-structures comprises one or more guidance-features configured to guide the growth of the plurality of cells; and
one or more interface elements associated with each of the tissue-engagement-structures, each interface element configured to couple the one or more of the plurality of cells.

A further aspect of the invention is a use of the bio-electronic interface described herein for treating and/or preventing a condition associated with one or more neuronal deficiency. To describe that aspect of the invention in a different way, the invention includes a use of a plurality of cells in the manufacture of a bio-electronic interface for treating and/or preventing a condition associated with one or more neuronal deficiency, wherein the bio-electronic interface comprises:
a substrate;
a plurality of micro-scale tissue-engagement-structures coupled to the substrate, in which each tissue-engagement-structures comprises one or more guidance-features configured to guide the growth of the plurality of cells; and
one or more interface elements associated with each of the tissue-engagement-structures, each interface element configured to couple the one or more of the plurality of cells.

An additional aspect of the invention is a method of treating or preventing a condition (such as a condition associated with one or more neuronal deficiency) in a subject in need thereof comprising administering the bio-electronic interface described herein to the subject or introducing the bio-electronic interface described herein into the subject.

A condition associated with one or more neuronal deficiency includes circumstances in which the subject has a reduced quality of life due to inadequacies in the way in which his/her neurons do, or do not, innervate cells and/or organs and/or tissues in the subject's body. In particular, this is any condition for which the subject's quality of life can be improved by using the bio-electronic interface described herein to form or improve neuronal connections, or to correct dysfunction in the nervous system of the subject. This includes situations in which neurons that are usually present are absent, where endogenous neurons have been removed, when neurons are insufficient in number to allow for correct function, where endogenous neurons are not functioning correctly (for example, where the function of the neurons has been altered by a viral infection or by a mutation) or the endogenous neurons are not functioning (for example, where the neurons have been damaged, perhaps by a trauma), and/or where endogenous neurons are innervating the incorrect cells and/or organs and/or tissues.

Accordingly, in one embodiment the condition associated with one or more neuronal deficiency is one or more condition selected from the list comprising: a reduced sensation (such as reduced vision, reduced smell, reduced hearing, a reduced pain-threshold, a reduced control of a limb(s) and/or a reduced sense of touch); an absent sensation (such as blindness, deafness, an absence of smell, an absence of pain, an absence of the control of a limb(s), and/or an absence of touch); a reduced control of a limb and/or a muscle (such as a reduced control of the sphincter); an absent control of a limb and/or a muscle (such as an absent control of the sphincter and/or incontinence); erroneous organ function and/or inflammation (such as asthma, Irritable Bowel Syndrome (IBS), Crohn's Disease and/or incontinence); a disease (such as endocrine disorders (including hypertension), Multiple Sclerosis (MS), and epilepsy); and a trauma (such as physical trauma).

An absent sensation may include circumstances in which a sensation is to be modulated, such as to introduce neuronal connections to prosthetic tissues and/or prosthetic organs and/or prosthetic limbs. This can include using the interface to allow the subject to have a sense of touch and/or a sense of pain and/or control in a prosthetic limb(s), and/or to allow the subject to have vision from an eye prosthesis.

In an alternative embodiment, the bio-electronic interface described herein can be utilised for non-medical uses, such as research (for example, functional mapping of neural circuitry or augmentation of a subject (including augmenting neural activity in the brain to annotate perception and cognition), memory enhancement, integration of machine learning, and/or integration of information databases with the nervous system).

The subject can be a mammalian subject or a non-mammalian subject. In one embodiment, the mammalian subject is a primate (such as a human, a monkey (including a rhesus macaque), or an ape (including a chimpanzee)), an equine (such as a horse), a bovine, a camel, a pig, a llama, an alpaca, a sheep, a goat, a canine, a feline, a rabbit, or a rodent (such as a mouse, guinea pig or rat). In one embodiment, the non-mammalian subject is avian (including a chicken), reptile, insect (including Drosophila melanogaster), fish (including Danio rerio), mollusc (including squid), or amphibian (including frogs such as Xenopus laevis). Preferably, the subject is a human.

It will be appreciated that the orientations described herein are often relative so, the terms “up” and “down” may be replaced by “down” and “up” in some case, excluding the cases where the effects of gravity are inherent to the working of the system. Similar considerations apply to similar terms, such as “top”, “bottom”, “left” and “right”.

1.-35. (canceled)

36. A bio-electronic interface comprising:
a substrate;
a plurality of micro-scale tissue-engagement-structures coupled to the substrate, in which each tissue-engagement-structure has a distal end providing a base on the substrate, in which each tissue-engagement-structures comprises one or more guidance-features configured to guide the growth of a plurality of cells; and
one or more interface elements associated with each of the tissue-engagement-structures, each interface element configured to be coupled to one or more of the plurality of cells.

37. The bio-electronic interface of claim 36, in which the plurality of micro-scale tissue-engagement-structures comprises an array of tissue-engagement-structures, in which one or more dividers are provided on the substrate to form a plurality of cell-separation-regions associated with the plurality of tissue-engagement-structures.

38. The bio-electronic interface of claim 36, in which the one or more guidance-features comprise one or more cavities within one or more of the tissue-engagement-structures.

39. The bio-electronic interface of claim 38, in which the one or more cavities each extend along the respective tissue-engagement-structures in a longitudinal direction.

40. The bio-electronic interface of claim 39, in which cavities associated with a particular tissue-engagement-structure laterally extend across the substrate by different extents.

41. The bio-electronic interface of claim 39, in which a divider separates a first cavity from a second cavity within a particular tissue-engagement-structure in which each cavity has a proximal opening and a distal opening, and the proximal opening of the first cavity is at a different longitudinal position to the proximal opening of the second cavity.

42. The bio-electronic interface of claim 38, in which the one or more guidance-features comprise striations within the one or more cavities.

43. The bio-electronic interface of claim 36, in which the one or more guidance-features comprise striations on an exterior of one or more of the tissue-engagement-structures.

44. The bio-electronic interface of claim 36, the distal end having a width of one of 10, 25, 50, 100 microns or less.

45. The bio-electronic interface of claim 44, in which each base has one or more buttresses configured to support the tissue-engagement-structure that extends from the base in which the buttresses comprise buttress-guidance-structures configured to guide the growth of the one or more cells towards the guidance-structures of the micro-needle.

46. The bio-electronic interface of claim 45, in which each tissue-engagement-structure extends transverse to the base in a longitudinal direction in which the base of one or more of the tissue engagement structures provides an offset in the longitudinal direction and supports a cover of the tissue engagement structures.

47. The bio-electronic interface of claim 36, in which each tissue-engagement-structure is needle-like.

48. The bio-electronic interface of claim 36, in which the substrate comprises a reservoir for the plurality of cells in which the substrate comprises one or more apertures to allow cells to pass between the reservoir and a surface of the substrate on which the bases of the tissue-engagement-structures are provided.

49. The bio-electronic interface of claim 36, in which one or more of the tissue-engagement-structures comprises a helical core.

50. The bio-electronic interface of claim 36, in which the tissue-engagement-structures are composed of degradable or partially degradable material.

51. A method of implanting a bio-electronic interface into an organ or a tissue, comprising:
receiving the bio-electronic interface of claim 1;
introducing the substrate to the organ or the tissue;
allowing formation of connections between the plurality of cells of the interface and the organ or the tissue in which the introduction is performed ex vivo.

52. A method of manufacturing a bio-electronic interface, comprising:
providing a substrate;
forming a plurality of micro-scale tissue-engagement-structures, in which each tissue-engagement-structure has a distal end providing a base on the substrate, in which each tissue-engagement-structures comprises one or more guidance-features configured to guide the growth of a plurality of cells; and
associating one or more interface elements with each of the tissue-engagement-structures, each interface element configured to be coupled to one or more of the plurality of cells.

53. The method of claim 52, further comprising applying the plurality or cells or a cell culture comprising the plurality of cells to the substrate.

54. The method of claim 53, in which the plurality of cells or the cell culture is applied during the step of forming the plurality of micro-scale tissue-engagement-structures on the substrate.

55. The method of claim 52, in which the tissue-engagement-structures are formed using two-photon lithography.
